(19) United States
(12) Patent Application Publication
(10) Pub. No.: US 2002/0143248 A1
(43) Pub. Date: Oct. 3, 2002

(54) MR microscopy of whole specimens, individual organs or tissue specimens perfused simultaneously with fixative and MR contrast agent

(76) Inventors: G. Allan Johnson, Chapel Hill, NC (US); Laurence W. Hedlund, Chapel Hill, NC (US)

Correspondence Address: NIXON & VANDERHYE P.C., 8TH Floor, 1100 North Glebe Road, Arlington, VA, 22201, US

(21) Appl. No.: 09/824,562

(22) Filed: Apr. 3, 2001

(51) Int. Cl. A61B 5/05

(52) U.S. Cl. 600/420

(57) ABSTRACT

Whole body animal specimens (preferably small mammals, such as laboratory rats, mice and the like) may be prepared for enhanced MR microscopy. Preferably, whole body animal specimens may be prepared for magnetic resonance (MR) microscopy by perfusing a mixture of a fixative and a MR contrast agent intravascularly of the animal specimen. The MR contrast agent is preferably a gadolinium compound, while the fixative is preferably formalin. In especially preferred embodiments, methods are provided whereby whole body animal specimens may be prepared for magnetic resonance (MR) microscopy by perfusing a MR contrast agent intravascularly of the animal specimen sequentially (a) into a jugular vein and out a carotid artery of the specimen, (b) into a carotid artery and out a jugular vein of the specimen, (c) into a carotid artery and out a femoral artery of the specimen; and then (d) into a jugular vein and out a femoral vein of the specimens.

### GOVERNMENT RIGHTS STATEMENT

This invention was made with Government support under Grant No. P41 RR05959 awarded by the National Institutes of Health. The Government has certain rights in the invention.

### FIELD OF THE INVENTION

The present invention relates generally to the field of magnetic resonance (MR) imaging. More specifically, the present invention relates to methods by which whole specimens may be rendered more susceptible to MR microscopy.

### BACKGROUND AND SUMMARY OF THE INVENTION

Traditional methods of studying internal anatomical structures rely on time-consuming light microscopy and histological sectioning. In this regard, specimens are typically dehydrated, stained, embedded and sectioned in preparation for studies. To obtain three-dimensional information, the sectioned material is typically photographed, traced, registered and then reconstructed into the whole specimen either mechanically for physical models or in a computer for visual models.

MR microscopy is a relatively recent extension of clinical magnetic resonance imaging (MRI) to maximize the resolution of small structures. See, Johnson et al, Magn. Reson. Quart., 9:1-30 (1993), the entire content of which is incorporated hereinto expressly by reference. It is a non-invasive and distortion-free technique for providing three dimensional histological information on whole specimens because it uses radio frequency (RF) energy to probe the water binding characteristics of the tissues. There is no need for physical sectioning the specimen, the registration of the slices is unnecessary since the specimen is imaged whole, and the process is extremely fast when compared to traditional histology and reconstruction.

It is known that whole specimens to be studied via MR microscopy may be perfused with an MR contrast agent (e.g., gadolinium chloride) subsequent to fixation. Smith et al, Proc. Natl. Acad. Sci., 91:3530-3553 (1994), the entire content of which is incorporated expressly hereinto by reference. According to this prior technique, the specimen may be fixed by initial perfusion with formalin fixative, followed by subsequent perfusion with a MR contrast agent, such as gadolinium DTPA (Gd), dissolved in a gelatin solution.

While perfusion of a specimen with a MR contrast agent subsequent to specimen fixation yields MR images with improved contrast in the vessels, improvements in the degree of contrast in the tissues fed by the vessels are still desired. It is towards providing such improvements to MR image contrast that the present invention is directed.

Broadly, the present invention is directed towards methods whereby whole body animal specimens (preferably small mammals, such as laboratory rats, mice and the like) or specific organs under study may be prepared for enhanced MR microscopy. More specifically, in preferred embodiments of the invention, methods are provided whereby a whole body animal specimen, isolated organ or other tissue specimen may be prepared for magnetic resonance (MR) microscopy by perfusing a mixture of a fixative and a MR contrast agent intravascularly in the animal specimen. Most preferably, the MR contrast agent is a gadolinium compound or other MR contrast agent, while the fixative may be formalin, gluteraldehyde, Bouin's solution, alcohol or other tissue fixative.

In especially preferred embodiments of the invention, methods are provided whereby whole body animal specimens may be prepared for magnetic resonance (MR) microscopy by perfusing a MR contrast agent intravascularly of the animal specimen sequentially (a) into a jugular vein and out a carotid artery of the specimen, (b) into a carotid artery and out a jugular vein of the specimen, (c) into a carotid artery and out a femoral artery of the specimen; and then (d) into a jugular vein and out a femoral vein of the specimen.

Other aspects and advantages of the present invention will become more clear after careful consideration is given to the following detailed description of the preferred exemplary embodiments thereof.

### DETAILED DESCRIPTION OF THE INVENTION

According to certain preferred embodiments of the present invention, a whole body animal specimen may be prepared for MR microscopy by the intravascular perfusion of a mixture of a chemical fixative and a MR contrast agent. The mixture will most preferably be comprised of a volumetric ratio of fixative to MR contrast agent varying from about 100:1 to about 1:1, and typically about 10:1.

Preferred chemical fixatives include Buffered formalin, glutaraldehyde, alcohol, picric acid and Bouin's solution An especially preferred chemical fixative that may be employed in the practice of the present invention is formalin.

A variety of MR contrast agents may be employed in the practice of the present invention, such as Gd-DTPA (Magnevist, Berlex), gadoteridol (Prohance, Bracco Diagnostics), gadomere (Gd-dendrimer, Schering AG). Most preferably, the MR contrast agent includes a stable free radical such asgadolinium. Clinically available MR contrast agents such as Prohance, and Magnevist are readily available. More selective free radical agents, e.g. organic free radicals that are not routinely considered for clinical use may be appropriate for these uses since toxicity is not an issue.

The present invention will be further understood by reference to the following non-limiting Example.

### EXAMPLE

Eight (8) mice were examined: four (4) wild type C57BL/6J and four (4) Uox −/−homozygotes on a hybrid of C57BL/6J-129SV. Half of each strain were about 2-months old and others were about 2-years old. These animals were prepared for whole body systemic perfusion fixation and staining by first placing catheters in the right external jugular vein and left carotid artery while under pentobarbital anesthesia. Heparin in saline was infused IV. Animals were then sacrificed by anesthetic overdoes.

Subsequently, the following four-step systemic perfusion sequence was followed using a mixture of 10% formalin and Magnevist (Berlex, gadopentetate dimeglumine) in a ratio of 10:1 (V:V): (1) perfusion into the jugular and out the carotid for the thorax; (2) into the carotid and out the jugular veins for the head, (3) into the carotid and out the femoral artery for abdominal arterial system, and (4) into the jugular vein and out the femoral vein for the abdominal veins.

For imaging, the whole body was placed in an acrylic sample holder (3-cm dia.×13-cm length) surrounded by an a proton perfluorcarbon material (fomblin) and imaged in a solenoid coil in a 2 T (30-cm-dia. bore) and 7 T system (15-cm-dia bore). These systems were controlled by GE Signa consoles running Epic version 5.7. An in-house version of 3D spin-warp imaging was used (TR 50 msec, TE 5 msec). In the 2T system, whole boy images were obtained with a resolution of 110 microns isotropic (array size 256×256×1024, FOV 113 mm), and in the 7 T system, the upper abdomen was selectively imaged with voxels 50×50×50 microns. Image array size was 512×512×512. Data were reconstructed on a SGI workstation and visualized using VoxelView (Vital Images, MN).

The use of GD in the perfusion mixture not only improved SNR, but also greatly shortened imaging time as compared to formalin perfusion alone. Imaging times for the whole body and selective regions were about 14 hr each. Furthermore, Gd remained intravascularly in high enough concentration to provide excellent vascular definition for several days following perfusion. Isotropic images (110-micron voxels) of the whole body shoed excellent vascular detail, especially in the lungs, liver and kidneys. Selective volumetric images (50-micron voxels) of the upper abdomen showed exquisite structural detail of the liver, stomach, spleen, kidneys, small intestines and large abdominal vessels.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method of preparing a whole body animal specimen, individual organs, or tissue specimens for magnetic resonance (MR) microscopy comprising perfusing a mixture of a fixative and a MR contrast agent intravascularly of an animal specimen.

2. The method of claim 1, wherein the MR contrast agent includes a stable free radical.

3. The method of claim 2, wherein the MR contrast agent includes a gadolinium compound.

4. The method of claim 1, wherein the fixative includes formalin.

5. The method of claim 1, wherein the mixture contains the fixative and MR contrast agent in a ratio of about 10:1.

6. The method of claim 1, wherein said step of perfusion includes perfusion of the mixture into a jugular vein and out a carotid artery of the specimen.

7. The method of claim 1, wherein said step of perfusion includes perfusion of the mixture into a carotid artery and out a jugular vein of the specimen.

8. The method of claim 1, wherein said step of perfusion includes perfusion of the mixture into a carotid artery and out a femoral artery of the specimen.

9. The method of claim 1, wherein said step of perfusion includes perfusion of the mixture into a jugular vein and out a femoral vein of the specimen.

10. The method of claim 1, wherein said step of perfusion includes perfusing the mixture sequentially:
(a) into a jugular vein and out a carotid artery of the specimen;
(b) into a carotid artery and out a jugular vein of the specimen;
(c) into a carotid artery and out a femoral artery of the specimen; and then
(d) into a jugular vein and out a femoral vein of the specimen.

11. A whole body MR microscopy method which comprises preparing a whole body animal specimen according to any one of claims 1-10, and then subsequently conducting MR microscopy of the prepared whole body animal specimen.

12. A method of preparing a whole body animal specimen for magnetic resonance (MR) microscopy comprising perfusing a MR contrast agent intravascularly of the animal specimen:
(a) into a jugular vein and out a carotid artery of the specimen;
(b) into a carotid artery and out a jugular vein of the specimen;
(c) into a carotid artery and out a femoral artery of the specimen; and then (d) into a jugular vein and out a femoral vein of the specimen.

13. The method of claim 12, wherein steps (a) through (d) are practiced sequentially.

14. The method of claim 12 or 13, wherein the MR contrast agent is perfused as a mixture with a fixative.

15. The method of claim 14, wherein the MR contrast agent includes a stable free radical compound.
